(19) United States
(12) Patent Application Publication
(10) Pub. No.: US 2026/0115489 A1
(43) Pub. Date: Apr. 30, 2026

(54) HANDHELD PHOTOTHERAPY DEVICE

(71) Applicant: SHENZHEN NUON MEDICAL EQUIPMENT CO., LTD., Shenzhen (CN)

(72) Inventors: Alain Dijkstra, Amstelveen (NL); Li Xiang, Shenzhen (CN); Tang Yuanzhou, Shenzhen (CN); Huang Jianlong, Shenzhen (CN); Mou Xiong, Shenzhen (CN)

(73) Assignee: Shenzhen Nuon Medical Equipment Co., Ltd., Shenzhen (CN)

(21) Appl. No.: 19/371,338

(22) Filed: Oct. 28, 2025

(30) Foreign Application Priority Data
Oct. 30, 2024 (CN) 2024226468799
Dec. 13, 2024 (CN) 2024231001832

(51) Int. Cl. A61N 5/06; A61H 15/00

(52) U.S. Cl. CPC A61N 5/0616; A61H 15/0078; A61H 2015/0042; A61H 2201/0153; A61N 2005/0644; A61N 2005/0664

(57) ABSTRACT

Embodiments of the present invention provide a multifunctional handheld skincare device combining phototherapy, massage, and microcurrent stimulation. The device includes a handle housing, a phototherapy head with dual light-transmitting surfaces and corresponding light-emitting elements, at least one massage element rotatably coupled to the handle, and at least one microcurrent stimulation element electrically connected to a circuit board. The phototherapy head emits light onto the skin, the massage element provides mechanical stimulation, and the microcurrent element delivers controlled electrical signals. This integrated configuration enables simultaneous or sequential application of phototherapy, massage, and microcurrent treatments, reducing manual adjustments, enhancing convenience, and improving therapeutic outcomes for skincare and cosmetic treatments.

### TECHNICAL FIELD

The present invention relates to the field of handheld beauty and therapeutic devices. More particularly, the invention relates to a multifunctional handheld device that combines phototherapy with massage, microcurrent stimulation, or other treatment components for improved skincare and therapeutic outcomes.

### BACKGROUND ART

In recent years, personal skincare and therapeutic devices have gained significant popularity, driven by growing consumer awareness of skin health, aesthetics, and wellness. Handheld beauty instruments, in particular, have become an accessible solution for home and professional use, offering treatments such as phototherapy, massage, microcurrent stimulation, and other physical therapies. Phototherapy devices, for instance, utilize specific wavelengths of light, such as red, blue, or near-infrared, to penetrate the skin and stimulate cellular activity, enhance local blood circulation, promote collagen synthesis, or accelerate healing of damaged tissue. Massage devices, on the other hand, provide mechanical stimulation to relieve muscle tension, improve blood flow, and promote lymphatic drainage, while microcurrent devices deliver low-intensity electrical currents to activate facial muscles and improve skin tone.

Despite their benefits, conventional devices typically suffer from functional and ergonomic limitations. Phototherapy devices often emit light from a single surface, forcing users to repeatedly adjust the device to ensure uniform coverage across different skin areas. This requirement is particularly inconvenient when treating areas such as the face, neck, or limbs, where multiple angles and orientations are needed. Likewise, traditional handheld massagers generally include a fixed or non-removable massage head, which can accumulate oils, lotions, or skin debris during use. This makes cleaning difficult and may affect hygiene, limiting frequent or shared use of such devices.

Attempts have been made to create multifunctional handheld instruments that combine phototherapy, massage, or other therapeutic modalities. However, these devices frequently introduce new challenges, such as increased size, weight, or complexity. Devices with only single-surface light emission cannot accommodate changes in hand orientation or reverse grips, reducing usability and flexibility. Additionally, integration of microcurrent or skin detection features often requires careful arrangement of internal electronics and power sources, which conventional designs fail to optimize. Consequently, many multifunctional devices still compromise on ergonomics, ease of maintenance, or the quality and consistency of therapeutic effects.

There remains a need for a compact, ergonomically designed handheld device that can provide multiple therapeutic modalities simultaneously while minimizing operational limitations. Such a device should enable dual-sided phototherapy to reduce frequent adjustments, incorporate a massage interface that is easy to clean and resistant to residue accumulation, and optionally provide microcurrent stimulation or skin monitoring for enhanced skin care outcomes. Moreover, the device should be lightweight, user-friendly, and adaptable to various hand grips and orientations to maximize convenience and therapeutic effectiveness.

The present invention addresses these needs by providing a multifunctional handheld device that integrates dual-sided phototherapy, an easily cleanable massage mechanism, and additional treatment features in a single, ergonomic unit. By combining these capabilities, the device offers enhanced usability, improved therapeutic outcomes, and a more efficient and hygienic user experience compared to conventional handheld beauty and therapeutic instruments.

### OBJECTS OF THE INVENTION

Some of the objects of the invention are as follows:

An object of the present invention is to provide a handheld multifunctional phototherapy device that combines dual-sided light-emitting phototherapy with a massage mechanism and optional microcurrent stimulation to deliver enhanced skincare and therapeutic outcomes.

Another object of the invention is to provide a device that emits light from two opposing surfaces, allowing the user to switch hand orientations or treat different areas of the skin without repeatedly adjusting the device, thereby improving usability and convenience.

A further object of the invention is to provide a massage mechanism integrated with the handheld device that is easy to clean and prevents the accumulation of oils, lotions, or debris, ensuring hygiene and maintenance convenience.

Yet another object of the invention is to provide a handheld device with microcurrent electrodes and skin detection capabilities, enabling additional skin treatments, monitoring, or stimulation in conjunction with phototherapy and massage.

An additional object of the invention is to provide a compact, lightweight, and ergonomic design, which allows the device to be comfortably held and operated in various hand grips, including forward and reverse grips, while maintaining effective contact with the skin.

Another object of the invention is to provide a multifunctional device with integrated electronics and power management, including a circuit board, rechargeable battery, and optional wireless communication modules, ensuring safe, efficient, and reliable operation.

A further object of the invention is to provide a versatile device adaptable to different therapeutic modalities, such as red, blue, or infrared light therapy, mechanical massage, and microcurrent stimulation, allowing a single device to address multiple skincare or therapeutic needs.

Yet another object of the invention is to provide a device that combines hygiene, usability, and multifunctionality, overcoming the limitations of conventional handheld beauty instruments that require repeated adjustments, are difficult to clean, or provide only single-modality treatment.

### SUMMARY OF THE INVENTION

According to a first aspect of the present invention, a handheld device is provided. The handheld device comprising: a handle housing configured to be gripped by a user; a phototherapy head disposed at one end of the handle housing, the phototherapy head having a first light-transmitting surface and a second light-transmitting surface disposed opposite each other; and a first light-emitting element and a second light-emitting element disposed within the phototherapy head, the first light-emitting element emits light facing the first light-transmitting surface and the second light-emitting element emits light facing the second light-transmitting surface.

In one embodiment of the invention, the phototherapy head is flat, the first light-transmitting surface and the second light-transmitting surface are spaced apart along the thickness of the head, and the distance between the first light-transmitting surface and the second light-transmitting surface gradually decreases as the distance away from the handle housing is increased.

In one embodiment of the invention, the first light-emitting element comprises a first light board and a first light-emitting unit fixed to the first light board, and the second light-emitting element comprises a second light board and a second light-emitting unit fixed to the second light board; the first light board is parallel to the first light-transmitting surface, and the second light board is parallel to the second light-transmitting surface.

In one embodiment of the invention, the handheld device further comprising at least one stimulation element electrically connected to a circuit board, the at least one stimulation element is selected from a group consisting of but is not limited to a heating element, a cooling element, a Peltier element, a microcurrent electrode, a piezoelectric element, a vibration element, or a massage element.

In one embodiment of the invention, the microcurrent electrode has a proximal end and a distal end, the proximal ends being closer to each other and closer to the end of the phototherapy head opposite the handle housing relative to the distal ends, and the distal ends being spaced apart from each other.

In one embodiment of the invention, the handheld device further comprising two conductive strips, one end of each conductive strip being connected to the microcurrent electrode, the other end connected to the circuit board; the phototherapy head is a transparent plastic component, and the microcurrent electrodes, conductive strips, and phototherapy head are integrally injection molded.

In one embodiment of the invention, the handheld device further comprising a first bracket and a second bracket located within the handle housing and spaced apart along the length of the handle housing, the first bracket having a first slot and the second bracket having a second slot, one end of the circuit board inserted into the first slot and the other end into the second slot; the second bracket being fixed to the handle housing, and the first and second light-emitting elements being fixed to the first bracket.

In one embodiment of the invention, the handheld device further comprising two conductive members, a skin detection module, and a charging module, wherein one end of each conductive member is exposed from the handle housing or the phototherapy head, the other end connected to the skin detection module and the charging module, respectively, forming a skin detection circuit and a charging circuit.

According to a second aspect of the present invention, a handheld device is provided. The handheld device comprising: a housing comprising a handle, a rotating portion disposed at one end of the handle, and a mounting portion disposed at the other end of the handle; a circuit board disposed within the handle; at least one stimulation element disposed within the mounting portion and electrically connected to the circuit board; and a massage element disposed outside the housing, the massage element having a first mounting cavity, the first mounting cavity is open for insertion of the rotating portion rotatably engaging with the first mounting cavity.

In one embodiment of the invention, one of the rotating portions and the massage element is provided with a rotating shaft, and the other is provided with an axial hole, the rotating shaft having a shaft sleeve located within the axial hole.

In one embodiment of the invention, the rotating portion is provided with a screw hole, the rotating shaft having a threaded portion at one end and a stopper at the other end, the threaded portion screwed to the screw hole, and the stopper located on a side of the shaft hole away from the screw hole, with the radial dimension of the stopper being greater than the diameter of the shaft hole.

In one embodiment of the invention, a retaining groove is provided on a side of the massage element away from the handle, the axial hole extends through the bottom wall of the retaining groove, the retaining portion is located within the retaining groove, and the beauty device further comprises a sealing member disposed in the retaining groove to cover the retaining portion.

In one embodiment of the invention, a raised portion is provided on the bottom wall of the first mounting cavity, the axial hole extends through the raised portion and communicates with the first mounting cavity; a retaining rib is provided on the end surface of the rotating portion, and the retaining rib surrounds the outer circumference of the raised portion.

In one embodiment of the invention, the handheld portion is elongated and tapered in a direction from the rotating portion toward the mounting portion; the mounting portion is elongated, with the longitudinal direction of the mounting portion intersecting with the longitudinal direction of the handheld portion.

In one embodiment of the invention, the at least one stimulation element includes a light therapy component electrically connected to the circuit board, the mounting portion has a conductive surface with a second mounting cavity, and a light-transmitting member is disposed at the opening of the second mounting cavity, the light therapy component being disposed within the light-transmitting member.

In one embodiment of the invention, the at least one stimulation element comprises two micro-current electrodes for providing microcurrent therapy.

According to a third aspect of the present invention, a method for providing a therapy to a user is provided. The method comprising: providing a handheld device comprising a handle housing, a phototherapy head having a first light-transmitting surface and a second light-transmitting surface disposed opposite each other, at least one massage element rotatably coupled to the handle; gripping the handle housing; emitting light from a first light-emitting element toward the first light-transmitting surface and from a second light-emitting element toward the second light-transmitting surface; bringing at least one of the light-transmitting surfaces into contact with or proximity to a target skin area; and moving the massage element over the target skin area.

In one embodiment of the invention, the massage element is rotatably coupled to a rotating portion disposed at one end of the handle, the method comprising rotating the rotating portion to cause the massage element to move over the target skin area.

In one embodiment of the invention, the method further comprising: providing one or more microcurrent electrodes in the handheld device and forming a closed circuit through the skin to deliver microcurrent to the target area.

In one embodiment of the invention, the method further comprising: adjusting the orientation of the handle to selectively bring either the first or second light-transmitting surface into contact with the skin without repositioning the massage element.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “handheld therapy device” refers to any device configured to emit therapeutic light for skin treatment, pain relief, or wellness applications.

In the context of the specification, the term “stimulation element” refers broadly to any component, module, or structure configured to apply a therapeutic or cosmetic stimulus to a user's skin or tissue. Stimulation elements may include, but are not limited to, phototherapy elements, massage elements, microcurrent electrodes, ultrasonic transducers, heating elements, cooling elements, or combinations thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “head” or “phototherapy head” refer to a portion of the device coupled to the housing and configured to emit light toward the skin. The head may include one or more light-transmitting surfaces, optical lenses, or diffusers, and may also support electrodes or other stimulation elements.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 is a schematic structural diagram of a handheld therapy device and a charging station, in accordance with an embodiment of the present invention.

FIG. 2 shows a perspective view of the handheld therapy device, in accordance with an embodiment of the present invention.

FIG. 3 is a front view of the handheld therapy device of FIG. 2, in accordance with an embodiment of the present invention.

FIG. 4 shows a cross-sectional view of the handheld therapy device along the A-A line of FIG. 3, in accordance with an embodiment of the present invention.

FIG. 5 shows an enlarged view of region B of the handheld therapy device of FIG. 4, in accordance with an embodiment of the present invention.

FIG. 6 shows an enlarged view of region C of the handheld therapy device of FIG. 4, in accordance with an embodiment of the present invention.

FIG. 7 is a top view of the handheld therapy device, in accordance with an embodiment of the present invention.

FIG. 8 shows a cross-sectional view of the handheld therapy device taken along line D-D of FIG. 7, in accordance with an embodiment of the present invention.

FIG. 9 shows an exploded view of the top portion of the handheld therapy device showing a microcurrent electrode, in accordance with an embodiment of the present invention.

FIG. 10 is a schematic diagram illustrating the structure of the microcurrent electrode of FIG. 9, in accordance with an embodiment of the present invention.

FIG. 11 is a schematic diagram illustrating an internal structure of the handheld therapy device, in accordance with an embodiment of the present invention.

FIG. 12 is a schematic diagram of the structure of a first bracket of the handheld therapy device, in accordance with an embodiment of the present invention.

FIG. 13 is a bottom perspective view of the first bracket, in accordance with an embodiment of the present invention.

FIG. 14 is a schematic diagram of the structure of a second bracket of the handheld therapy device, in accordance with an embodiment of the present invention.

FIG. 15 illustrates the handheld therapy device showing a massage component provided on an end opposite to a phototherapy component, in accordance with an embodiment of the present invention.

FIG. 16 is a cross-sectional view of the handheld therapy device of FIG. 15, in accordance with an embodiment of the present invention.

FIG. 17 shows an enlarged view of region A of the handheld therapy device of FIG. 16, in accordance with an embodiment of the present invention.

FIG. 18 illustrates a perspective exploded view of the handheld therapy device in accordance with an alternate embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a multifunctional handheld device that integrates a plurality of stimulation elements, including phototherapy stimulation, massage stimulation, and microcurrent stimulation, within a compact and ergonomic housing. The device is designed to enhance skin health, improve user convenience, and combine multiple treatment modalities into a single portable unit.

The device comprises a main housing configured to be comfortably held in a user's hand. The housing accommodates internal components such as a rechargeable power supply, control circuitry, and signal processing elements. The ergonomic shape ensures ease of use across different regions of the face and body.

In one embodiment, a phototherapy stimulation element is disposed within the housing or within a phototherapy head connected to the housing. The phototherapy stimulation element may include one or more light-emitting diodes (LEDs), laser diodes, or equivalent light sources, configured to emit light of predetermined therapeutic wavelengths. The phototherapy head may comprise dual light-transmitting surfaces, allowing the stimulation element to emit light from opposite sides, thereby reducing the need for frequent repositioning during treatment.

In another embodiment, the device includes a massage stimulation element. The massage stimulation element may comprise one or more rotatable rollers, kneading members, or vibrating units disposed outside the housing. These elements apply mechanical stimulation to the skin, enhancing blood circulation, relieving muscular tension, and promoting deeper penetration of skincare formulations. The massage stimulation element may be removably mounted on a rotating portion of the housing to facilitate cleaning and prevent the accumulation of impurities.

The device further comprises a microcurrent stimulation element, configured to deliver low-level electrical currents to the skin surface. The microcurrent stimulation element may include a pair of electrodes disposed on an external portion of the device and electrically connected to the circuit board. When activated, the electrodes generate a safe, controlled current that stimulates cellular activity, supports collagen synthesis, and enhances muscle tone.

The plurality of stimulation elements may be operated individually or in combination. For example, the phototherapy stimulation element and the massage stimulation element may be operated simultaneously to provide light therapy while mechanically stimulating the skin. In other cases, the microcurrent stimulation element may be activated concurrently with the phototherapy stimulation element to achieve synergistic benefits.

A control interface is provided on the housing, enabling the user to select between different modes of operation, such as phototherapy-only, massage-only, microcurrent-only, or combinations thereof. The control interface may include buttons, switches, or a touchscreen panel. In advanced embodiments, the control interface may also adjust intensity levels or treatment durations of each stimulation element.

The device may further incorporate safety and monitoring features. A skin-contact sensor may be operatively coupled to one or more stimulation elements to ensure that phototherapy or microcurrent stimulation is delivered only when the device is in proper contact with the skin. Additional sensors may monitor skin impedance, temperature, or treatment time to optimize operation and protect the user.

In certain embodiments, the stimulation elements may be modular or interchangeable. For example, the massage stimulation element may be detached and replaced with another form of stimulation element, such as a cooling head or ultrasonic applicator, while still maintaining compatibility with the same housing and circuitry.

The components of the device may be fabricated from biocompatible and durable materials. Transparent or translucent polymers may be employed for light-transmitting regions of the phototherapy head, while elastomers or metallic elements may be used for the massage stimulation element. Conductive alloys or coatings may be used for the electrodes of the microcurrent stimulation element to ensure safe and effective current delivery.

By integrating a plurality of stimulation elements within a single handheld therapy device, the invention addresses limitations of prior art devices that typically provide only one treatment modality. The multifunctional arrangement enhances convenience, reduces the need for multiple separate devices, and provides improved therapeutic outcomes through combined stimulation approaches.

Embodiments of the present invention will now be described with reference to the FIGS.

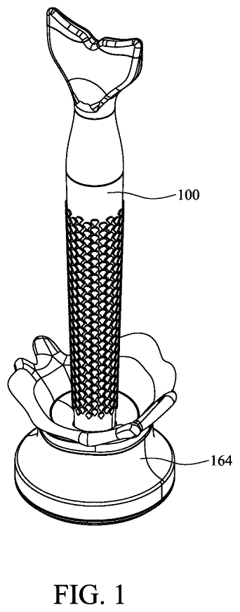

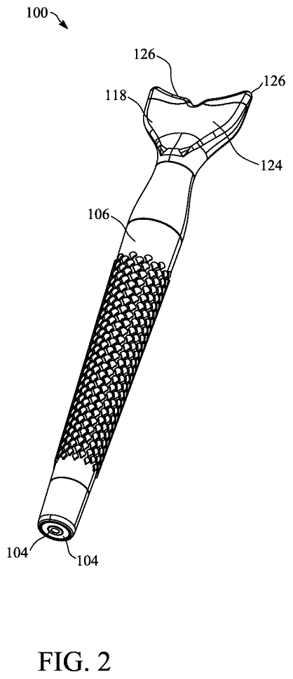

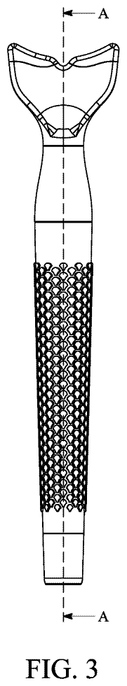

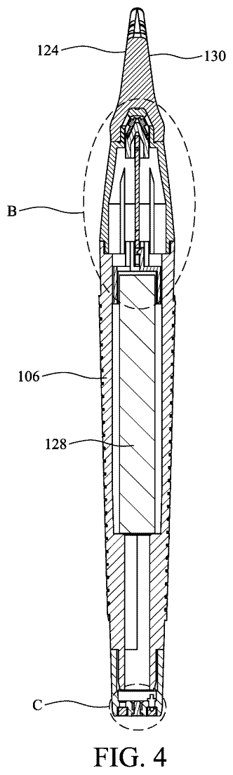

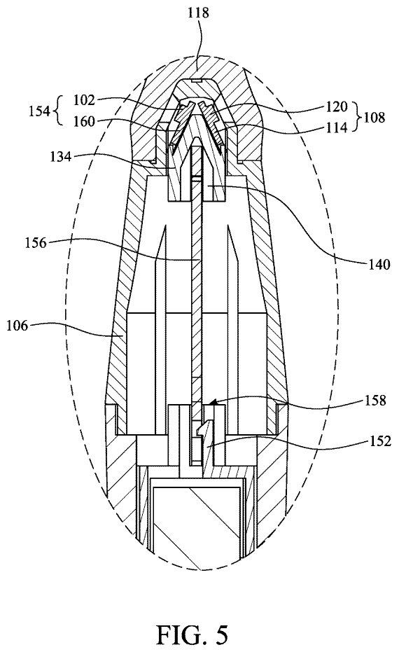

Referring to FIGS. 1 to 5, an embodiment of the present invention discloses a handheld therapy device 100. The handheld therapy device 100 comprises a handle housing 106, a phototherapy head 118, a first light-emitting element 154, and a second light-emitting element 108. The handle housing 106 is configured to be grasped by a user during operation. The phototherapy head 118 is disposed at one end of the handle housing 106 and includes a first light-transmitting surface 124 and a second light-transmitting surface 130 disposed on opposite sides of the head. The first light-emitting element 154 and the second light-emitting element 108 are positioned within either the handle housing 106 or the phototherapy head 118. The first light-emitting element 154 directs light toward the first light-transmitting surface 124, while the second light-emitting element 108 directs light toward the second light-transmitting surface 130.

Conventional phototherapy devices typically emit light through only one light-transmitting surface, requiring the user to carefully align the device with the skin. For example, when transitioning treatment from one side of the face to the other, the user must repeatedly reposition and reorient the device to ensure proper contact and illumination. Such frequent adjustments reduce ease of use and limit treatment efficiency. By contrast, in the present invention, both the first light-transmitting surface 124 and the second light-transmitting surface 130 are capable of emitting therapeutic light. This dual-surface configuration allows the user to utilize either side of the phototherapy head 118, thereby eliminating the need for precise alignment and repeated repositioning. Accordingly, the device provides greater flexibility, enhances user comfort, and facilitates seamless switching between left-hand and right-hand operation.

In an embodiment, the phototherapy head 118 is substantially flat, with the first light-transmitting surface 124 and the second light-transmitting surface 130 disposed on opposing broad faces of the head. The spacing between the first light-transmitting surface 124 and the second light-transmitting surface 130 may decrease as the head extends outward from the handle housing 106, thereby forming a compact structure that maximizes the exposed treatment area while maintaining a lightweight profile. The flat configuration allows the phototherapy head 118 to cover a relatively large skin surface area during use, improving treatment efficiency without sacrificing portability.

In some embodiments, the width of the phototherapy head 118 gradually increases as it extends away from the handle housing 106. For example, the lower end of the phototherapy head 118, adjacent to the handle housing 106, may be narrower than the upper end of the phototherapy head 118, thereby forming a tapered or flared profile. In another embodiment, the upper end of the phototherapy head 118 is contoured such that the central region is lower than the lateral ends, resulting in a fishtail-like configuration. Such geometric variations enable the device to better conform to different facial or body contours.

In further embodiments, when the phototherapy head 118 is fabricated from a relatively flexible polymeric or elastomeric material, the head can undergo slight elastic deformation, such as bending or flexing. This feature allows the head to adapt dynamically to variations in skin curvature, ensuring consistent contact and uniform light distribution across the treatment area.

In alternative embodiments, the phototherapy head 118 adopts other geometries, such as square, triangular, or circular.

In the illustrated embodiment, the first light-emitting element 154 and the second light-emitting element 108 are disposed either at the junction of the handle housing 106 and the phototherapy head 118, within the phototherapy head 118, or within the handle housing 106. In a specific configuration, the first light-emitting element 154 and the second light-emitting element 108 are positioned inside the handle housing 106 at its upper end. Light holes are defined in the handle housing 106 corresponding to the first light-emitting element 154 and the second light-emitting element 108, and the phototherapy head 118 is mounted on the upper end of the handle housing 106, covering the light holes to permit the emission of light toward the skin.

The first light-emitting element 154 includes a first light board 160 and a first light-emitting unit 102 fixed to the first light board 160. Similarly, the second light-emitting element 108 includes a second light board 114 and a second light-emitting unit 120 fixed to the second light board 114. The first light board 160 is oriented parallel to the first light-transmitting surface 124, and the second light board 114 is oriented parallel to the second light-transmitting surface 130. As used herein, the term parallel refers to parallel or substantially parallel alignment. The light-emitting units are light-emitting diodes (LEDs) capable of emitting wavelengths such as red, infrared, blue, or violet light, thereby providing targeted phototherapy. In this embodiment, the first light board 160 and the second light board 114 are arranged at an angle relative to each other, with their upper ends converging and their lower ends diverging, to optimize light distribution within the phototherapy head 118.

The handheld therapy device 100 further includes two microcurrent electrodes 126 and a circuit board 156 electrically connected to the electrodes. The microcurrent electrodes 126 are mounted on the phototherapy head 118. When the electrodes contact the skin, the skin acts as a conductive medium, completing a circuit comprising the skin, the microcurrent electrodes 126, and the circuit board 156. In operation, the microcurrent electrodes 126 function as discharge electrodes, delivering low-level electrical stimulation to the skin. The integration of the microcurrent electrodes 126 within the phototherapy head 118 positions them in proximity to the phototherapy treatment area, enabling simultaneous delivery of microcurrent stimulation and phototherapy for enhanced therapeutic effects.

The microcurrent electrodes 126 are formed of conductive materials and are fabricated in different geometries, including metal contacts, posts, sheets, or blocks, provided that they ensure reliable conduction.

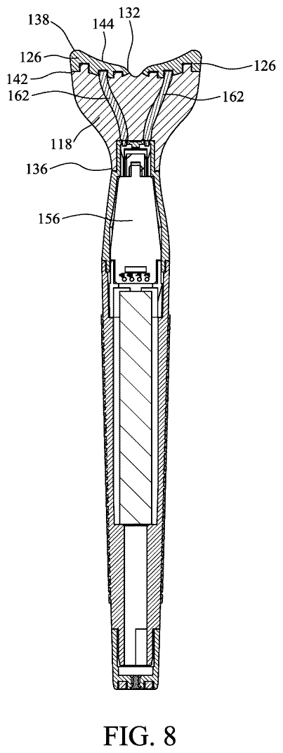

Referring to FIGS. 7 and 8, the phototherapy head 118 includes a first end 136 and a second end 142 disposed opposite to each other. The first end 136 is connected to the handle housing 106, and the first light-transmitting surface 124 and the second light-transmitting surface 130 extend longitudinally from the first end 136 to the second end 142. Two microcurrent electrodes 126 are fixed to the second end 142. Each microcurrent electrode 126 includes a proximal end 132 and a distal end 138. The proximal ends 132 of the electrodes are positioned closer to each other and closer to the second end 142 relative to the distal ends 138, while the distal ends 138 are spaced farther apart. This structural arrangement creates an overall contour where the middle portion of the phototherapy head 118 is lower than the lateral sides, thereby forming a topography that conforms closely to the curvature of the human face and ensures uniform treatment contact.

In one embodiment, these two microcurrent electrodes 126 contact surfaces are arranged in a V-shaped configuration, such that the apex of the “V” is oriented toward the longitudinal axis of the device. This V-shaped structure allows the electrodes to conform closely to the contour of the user's skin, particularly in curved regions such as the chin, jawline, cheeks, or neckline, thereby enhancing the effective area of electrical contact and improving the efficiency of microcurrent stimulation.

The V-shaped arrangement further assists in evenly distributing current flow between the two microcurrent electrodes 126 when the handheld therapy device is applied to the skin, resulting in a more uniform treatment effect. Moreover, the angled structure of the microcurrent electrodes 126 surfaces provides better fit and contact pressure when the device is moved along facial contours, improving comfort and stability during use.

In certain embodiments, the microcurrent electrodes 126 are elongated in the direction extending from the proximal ends 132 to the distal ends 138. Each microcurrent electrode 126 includes a contact surface 144 that connects the proximal ends 132 and distal ends 138 and faces away from the second end 142. The contact surface 144 is configured as a curved or inclined surface. In the present embodiment, the contact surface 144 constitutes the upper surface of the microcurrent electrode 126, which directly engages the user's skin during microcurrent stimulation. When the contact surface 144 is arc-shaped, it is formed either as a segment of constant curvature or as a surface generated by sequentially connecting multiple segments having different curvatures, thereby optimizing conformity to the skin profile.

The handheld therapy device 100 further includes two conductive strips 162. One end of each conductive strip 162 is electrically connected to a corresponding microcurrent electrode 126, while the opposite end of each conductive strip 162 is connected to the circuit board 156. The phototherapy head 118 is fabricated from a transparent plastic material, and the microcurrent electrodes 126, conductive strips 162, and phototherapy head 118 are integrally formed by injection molding. In this configuration, the microcurrent electrodes 126 and conductive strips 162 are embedded as inserts during molding, thereby securing them within the phototherapy head 118 and preventing separation during use. In a preferred embodiment, the phototherapy head is fabricate from liquid silicone through injection molding process.

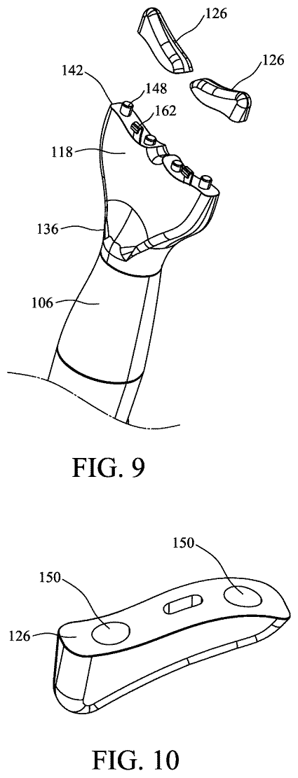

Referring to FIGS. 9 and 10, the bonding strength between the microcurrent electrodes 126 and the phototherapy head 118 is further enhanced by providing one or more engaging grooves 150 on the lower surface of each microcurrent electrode 126. During molding, molten material of the phototherapy head 118 fills the engaging grooves 150. After solidification, engaging protrusions 148 are formed at the second end 142, locking into the engaging grooves 150. This interlocking configuration increases the contact area between the microcurrent electrodes 126 and the phototherapy head 118, thereby improving structural stability and durability.

The majority of each conductive strip 162 is embedded within the phototherapy head 118, with only its upper and lower ends exposed to allow electrical connection with the microcurrent electrodes 126 and the circuit board 156, respectively. Since the phototherapy head 118 is transparent, regions not obstructed by internal components remain translucent, permitting light transmission. This structure ensures that a larger portion of the phototherapy head 118 contributes to light emission, thereby expanding the effective phototherapy treatment area.

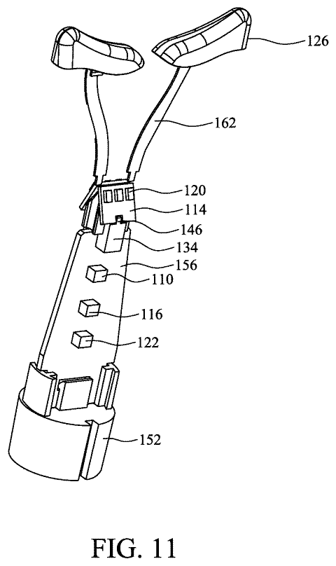

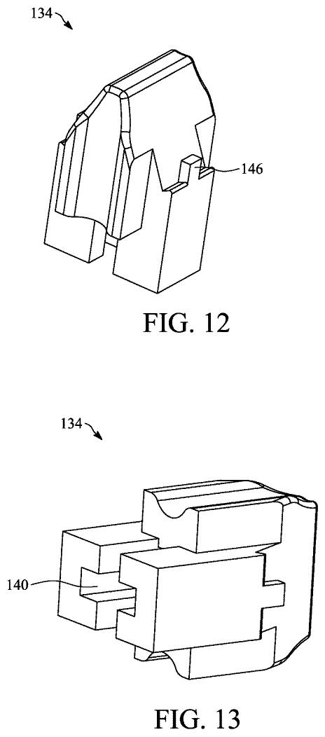

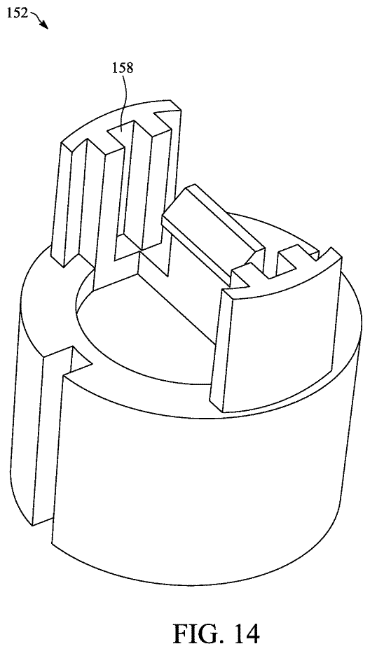

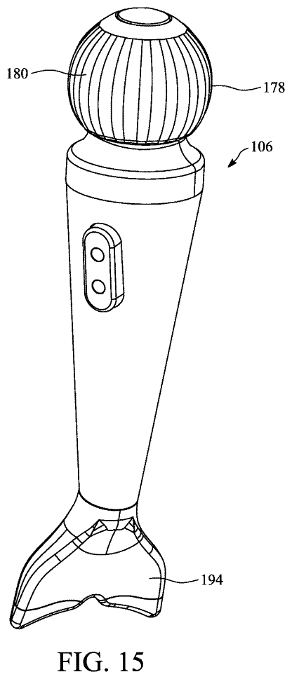

Referring to FIGS. 11 to 14, the handheld therapy device 100 includes a first bracket 134, a second bracket 152, and a circuit board 156. The first bracket 134 and the second bracket 152 are disposed within the handle housing 106 and spaced apart along its length. In one embodiment, the first bracket 134 is positioned above the second bracket 152.

The first bracket 134 is provided with a first slot 140, and the second bracket 152 is provided with a second slot 158. The first slot 140, and the second slot 158 are aligned opposite each other. One end of the circuit board 156 is inserted into the first slot 140, and the opposite end is inserted into the second slot 158. This arrangement secures the upper and lower ends of the circuit board 156, while the central portion remains unsupported, thereby facilitating heat dissipation. The second bracket 152 is fixed to the handle housing 106, and the first bracket 134 relies on the circuit board 156 and the second bracket 152 for support. In some embodiments, the first bracket 134 abuts the handle housing 106 to limit upward displacement.

The first light-emitting element 154 and the second light-emitting element 108 are fixed to the first bracket 134. The first bracket 134 includes two retaining protrusions 146, and the first light board 160 and the second light board 114 each include a corresponding retaining groove (not shown) that engages with the retaining protrusions 146.

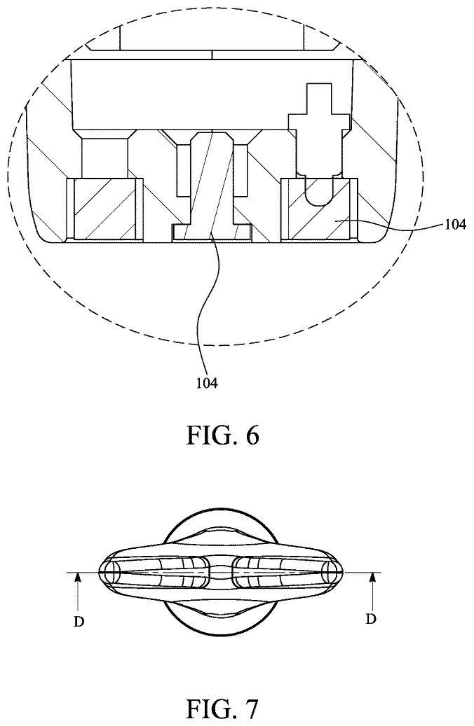

Referring to FIGS. 6 to 11, the device further includes two conductive members 104 and a charging module 110. One end of each conductive member 104 is exposed from the handle housing 106 or the phototherapy head 118, while the opposite end is electrically connected to the charging module 110, which is in turn connected to the circuit board 156. Together, the conductive members 104 and the charging module 110 form a charging circuit, with the conductive members 104 serving as positive and negative electrodes for charging a battery 128 located within the handle housing 106.

In one embodiment, one conductive member 104 is cylindrical, and the other is annular and disposed around the cylindrical conductive member 104. The conductive members 104 are mounted either on the handle housing 106 or on the phototherapy head 118. When mounted on the handle housing 106, the conductive members 104 are fixed at the bottom. In this configuration, charging is achieved by inserting the bottom of the handle housing 106 into a charging dock 164, where direct contact occurs between the conductive members 104 and the dock.

The handle housing 106 further incorporates a skin detection module 116. The conductive members 104 are electrically connected to the skin detection module 116, forming a skin detection circuit. In this configuration, the conductive members 104 act as detection probes, such as capacitance probes or resistance probes. When the skin surface containing oil and water contacts the conductive members 104, variations in dielectric constant, conductivity, and morphology alter the capacitance or resistance. These changes are detected and converted into electrical signals, allowing the determination of oil and water content in the skin. The skin itself functions as a conductor, thereby closing the skin detection circuit when contact is established.

In this embodiment, the conductive members 104 serve a dual function for both charging and skin detection. This dual-use configuration reduces the total number of conductive elements required, simplifies the device structure, and lowers manufacturing cost.

Furthermore, the handheld therapy device 100 includes a wireless communication module 122 fixed to the handle housing 106. The wireless communication module 122 is implemented as a Bluetooth module, an NFC module, or another wireless communication unit. The wireless communication module 122 is electrically connected to the skin detection module 116 and transmits information, including skin condition data collected by the conductive members 104, to an external terminal such as a mobile phone or computer. The charging module 110, the skin detection module 116, and the wireless communication module 122 are mounted on and electrically connected to the circuit board 156.

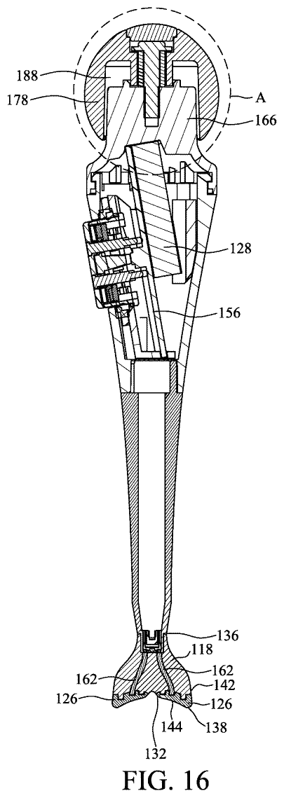

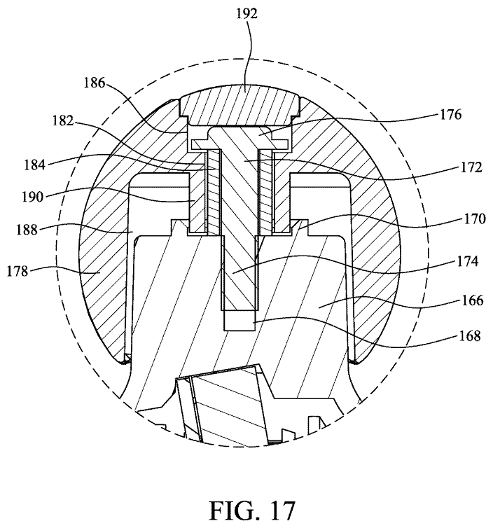

In an embodiment of the present invention, the handheld therapy device 100 further comprises a massage element mounted on a second end of the handheld therapy device, opposite to the phototherapy head 118. Referring to FIGS. 15 to 17, the handheld therapy device 100 comprises a handle housing 106, a circuit board 156, a phototherapy head 118, and a massage element 178. The handle housing 106 includes a rotating portion 166 positioned at one end and a mounting portion 194 positioned at the opposite end. The circuit board 156 is disposed within the handle housing 106, and the phototherapy head is disposed at the mounting portion 194 and electrically connected to the circuit board 156. The massage element 178 is positioned outside the handle housing 106 and defines a first mounting cavity 188 with an opening for receiving the rotating portion 166 to enable rotational movement.

The handle housing 106 is designed to be held by the user. During use, the user selects either the massage element 178 for skin massage or the phototherapy head 118 having one or more stimulation elements for skin treatment. The one or more stimulation elements include one or more of a microcurrent massage mechanism, a light therapy module, a heat compress module, or other therapeutic mechanisms. The outer surface of the massage element 178 forms a massage surface 180 surrounding the rotating portion 166. When the device is moved across the skin, the massage surface 180 directly contacts the skin, providing a rolling massage effect. The fully exposed arrangement of the massage element 178 ensures that only the massage surface 180 contacts the skin, preventing transfer of dirt and bacteria from other objects. This configuration simplifies cleaning, reduces contamination risk, and allows the massage element 178 to have a larger surface area for improved usability.

The massage element 178 is formed of metal or plastic and is shaped to facilitate smooth rolling. In one embodiment, the massage element 178 is spherical, while in other embodiments, it is ellipsoidal, cylindrical, or drum-shaped. The massage surface 180 optionally includes multiple ridges distributed circumferentially and extending axially along the rotating portion 166 to prevent slipping and enhance the massage effect. In other embodiments, the massage surface 180 is smooth.

The rotating connection between the rotating portion 166 and the massage element 178 is achieved through a rotating shaft 172 and a corresponding shaft hole 182. A sleeve 184 surrounds the rotating shaft 172 and is positioned within the shaft hole 182. The sleeve 184 is either fixed relative to the rotating shaft 172 and rotatable within the shaft hole 182, fixed within the shaft hole 182 and rotatable relative to the rotating shaft 172, or rotatable relative to both. The sleeve 184 is made of a wear-resistant material to increase durability. In one embodiment, both the sleeve 184 and the rotating shaft 172 are metal, providing high structural strength and wear resistance. In alternative embodiments, the sleeve 184 is made of a high-strength engineering plastic such as polyethylene, polypropylene, or nylon.

The rotating portion 166 is provided with a screw hole 168, and the rotating shaft 172 has a threaded portion 174 and a stopper 176. The threaded portion 174 engages with the screw hole 168, while the stopper 176 is positioned on the side of the shaft hole 182 opposite the screw hole 168. Because the radial dimension of the stopper 176 exceeds the diameter of the shaft hole 182, the massage element 178 is securely retained within the rotating portion 166. This configuration facilitates assembly and disassembly, allowing interchangeable massage elements 178.

A stopper slot 186 is defined on the side of the massage element 178 opposite the handle housing 106. The shaft hole 182 extends through the bottom wall of the stopper slot 186, and the stopper 176 is accommodated within the stopper slot 186, preventing excessive protrusion and ensuring smooth rotation. A sealing member 192 is positioned within the stopper slot 186 to enclose the stopper 176, with an outer surface flush with or raised relative to the outer surface of the massage element 178. The sealing member 192 is a soft rubber plug to prevent dirt accumulation.

The sleeve 184 extends beyond the length of the shaft hole 182, with one end abutting the stopper 176 and the other abutting the rotating portion 166. This arrangement prevents the stopper 176 from interfering with the rotation of the massage element 178 during assembly. A raised portion 190 is provided on the bottom wall of the first mounting cavity 188, through which the shaft hole 182 extends. The raised portion 190 is spaced from the inner circumference of the first mounting cavity 188, ensuring secure installation of the massage element 178.

A retaining rib 170 surrounds the outer circumference of the raised portion 190 and is positioned between the raised portion 190 and the screw hole 168. This prevents outward deformation of the raised portion 190 under rolling forces, ensuring stable rotation of the massage element 178 around the rotating shaft 172.

The handle housing 106 is elongated and tapered toward the mounting portion 194. The end of the handle housing 106 adjacent to the rotating portion 166 has an increased diameter to accommodate the circuit board 156, while the opposite end connects to the mounting portion 194, providing a compact yet strong structure. The mounting portion 194 is elongated in a direction intersecting the length of the handle housing 106, providing sufficient space for mounting therapeutic components while maintaining a generally flat device design.

In an embodiment of the present invention, the rotating portion further comprises at least one stimulation element electrically connected to a circuit board for providing one or more therapies to the user. The at least one stimulation element is selected from a group consisting of, but is not limited to a heating element, a cooling element, a Peltier element, a microcurrent electrode, a piezoelectric element, a vibration element, or a massage element.

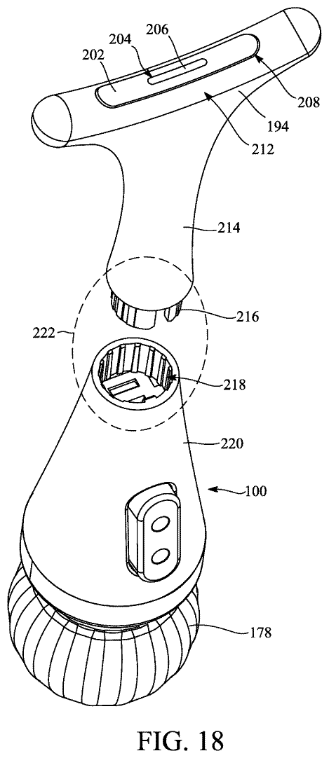

FIG. 18 illustrates a perspective exploded view of another configuration of the handheld therapy device 100 according to an embodiment of the present invention. The handheld therapy device 100 includes the housing 106, the massage component 178, and a therapeutic assembly positioned at the opposite end of the housing.

The housing 106 comprises a handheld portion 220, the mounting portion 194 extending from an upper end of the handheld portion 220, and the rotating portion 166 disposed at the lower end of the handheld portion 220 for connection with the massage component 178. The handheld portion 220 accommodates a control switch on its outer surface and houses an internal circuit board for electrical control of the device. The lower end of the housing is connected to a massage component 178, which serves as the contact and rolling component for performing skin massage therapy.

At the upper end of the handheld portion 220, the mounting portion 194 includes a mounting cavity 212 for housing one or more stimulation element 202, such as a light therapy or microcurrent treatment assembly. The mounting opening 208 on the upper side provides an exposed area for interaction with the skin. A protective cover 206 may be detachably mounted over the one or more stimulation element 202, which includes a transparent window 204 through which light or microcurrent is transmitted to the skin during operation.

The mounting portion 194 extends outward through a neck section 214 that is ergonomically curved for comfortable handling and use. The lower end of the neck section 214 includes engaging teeth 216 that mate with corresponding grooves 218 on the handheld portion 220 to form a detachable and rotatable connection, as indicated by 222. This structure facilitates the assembly and disassembly of the one or more stimulation element or mounting portion 194, enabling convenient replacement or maintenance.

The massage component 178 positioned at the lower end of the handheld therapy device provides a textured or ridged outer surface for improved skin contact and massaging performance. The rotational connection between the massage component 178 and the handheld portion 220 allows smooth rolling during use, enhancing the massage effect.

FIG. 18 depicts a multifunctional handheld therapy device incorporating both a therapeutic treatment assembly and a massage mechanism, wherein the detachable design of the mounting portion 194 and the ergonomic structure of the handheld portion 220 allow convenient operation, easy maintenance, and enhanced user comfort.

In an embodiment of the present invention, a method for providing a therapy to a user is disclosed, utilizing the handheld device as described in any of the foregoing embodiments. The method includes the steps of providing a handheld device comprising a handle housing, a phototherapy head, and at least one massage element. The phototherapy head includes a first light-transmitting surface and a second light-transmitting surface disposed opposite each other, while the handle housing contains a circuit board and associated control electronics for operating the therapeutic components. The massage element is rotatably coupled to a rotating portion located at one end of the handle housing.

In operation, the user grips the handle housing and positions the phototherapy head such that either the first light-transmitting surface or the second light-transmitting surface faces the desired skin treatment area. Upon activation, the device emits light from a first light-emitting element toward the first light-transmitting surface and from a second light-emitting element toward the second light-transmitting surface. The emitted light may include visible, infrared, or near-infrared wavelengths suitable for phototherapy or skin rejuvenation. The light energy penetrates the skin tissue to stimulate cellular activity, enhance collagen production, and improve local blood circulation. The user may bring either of the light-transmitting surfaces into direct contact with or in close proximity to the target skin area, depending on the treatment intensity desired.

In addition, the method may include moving the massage element over the target skin area while the phototherapy light is being emitted. The rotation of the massage element provides mechanical stimulation that promotes lymphatic drainage, reduces puffiness, and enhances the overall therapeutic effect. The massage element may be driven through manual movement of the device by the user, or by rotational coupling with the rotating portion of the handle housing. In some embodiments, the rotating portion can be actuated to produce a gentle oscillatory or rolling motion of the massage element, thereby increasing user comfort and improving treatment uniformity.

In some embodiments, the handheld device is equipped with one or more microcurrent electrodes configured to form a closed electrical circuit through the user's skin. The method may further comprise delivering low-intensity microcurrent to the target area by activating the microcurrent electrodes while maintaining skin contact. The microcurrent stimulation enhances cellular metabolism and promotes tissue repair when used in conjunction with light therapy or massage.

In another embodiment, the method includes adjusting the orientation of the handle housing to selectively bring either the first or the second light-transmitting surface into contact with the skin without detaching or repositioning the massage element. This allows the user to conveniently switch between two different light therapy zones, such as red light for collagen stimulation and near-infrared light for deeper tissue penetration, using the same device. The device may include control circuitry or a touch interface for switching between different modes corresponding to light therapy, massage, or microcurrent stimulation.

Through the combined use of phototherapy, microcurrent stimulation, and mechanical massage, the disclosed method provides a comprehensive multi-modal treatment capable of improving skin tone, texture, and elasticity, while also relieving localized muscular fatigue and enhancing relaxation.

The device of the present invention is particularly suited for use in the field of personal beauty care and home-based skin treatment. Owing to the integration of phototherapy, microcurrent stimulation, heat compress, and massage functionalities, the devices can be applied to promote skin rejuvenation, improve blood circulation, alleviate muscle fatigue, and enhance absorption of cosmetic products. Users may apply the device on the face, neck, and other body areas requiring cosmetic treatment, thereby reducing fine lines, improving skin elasticity, and restoring a youthful appearance.

Beyond cosmetic applications, the multifunctional structure of the devices allows for use in therapeutic and wellness settings. For example, the phototherapy and microcurrent components may be applied to relieve localized pain, accelerate skin recovery after minor dermatological procedures, and stimulate cell activity. The massage element further aids in lymphatic drainage, reducing puffiness, and relieving stress-related muscle tension, making the devices suitable for both preventive care and supplementary therapy.

In addition, the wireless communication module enables integration with mobile applications and smart terminals. This allows users to track skin condition, receive personalized treatment recommendations, and monitor therapy history. Accordingly, the invention is applicable not only in personal care and beauty salons but also in medical aesthetic clinics, wellness centers, and telehealth-based skincare management systems.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A handheld device comprising:
a handle housing configured to be gripped by a user;
a phototherapy head disposed at one end of the handle housing, the phototherapy head having a first light-transmitting surface and a second light-transmitting surface disposed opposite each other; and
a first light-emitting element and a second light-emitting element disposed within the phototherapy head, the first light-emitting element emits light facing the first light-transmitting surface and the second light-emitting element emits light facing the second light-transmitting surface.

2. The handheld device of claim 1, wherein the phototherapy head is flat, the first light-transmitting surface and the second light-transmitting surface are spaced apart along the thickness of the head, and the distance between the first light-transmitting surface and the second light-transmitting surface gradually decreases as the distance away from the handle housing is increased.

3. The handheld device of claim 1, wherein the first light-emitting element comprises a first light board and a first light-emitting unit fixed to the first light board, and the second light-emitting element comprises a second light board and a second light-emitting unit fixed to the second light board; and the first light board is parallel to the first light-transmitting surface, and the second light board is parallel to the second light-transmitting surface.

4. The handheld device of claim 1, further comprising at least one stimulation element electrically connected to a circuit board, the at least one stimulation element is selected from a group consisting of, but is not limited to a heating element, a cooling element, a Peltier element, a microcurrent electrode, a piezoelectric element, a vibration element, or a massage element.

5. The handheld device of claim 4, wherein the microcurrent electrode has a proximal end and a distal end, the proximal ends being closer to each other and closer to the end of the phototherapy head opposite the handle housing relative to the distal ends, and the distal ends being spaced apart from each other.

6. The handheld device of claim 5, further comprising two conductive strips, one end of each conductive strip being connected to the microcurrent electrode, the other end connected to the circuit board; and the phototherapy head is a transparent plastic component, and the microcurrent electrodes, conductive strips, and phototherapy head are integrally injection molded.

7. The handheld device of claim 4, further comprising a first bracket and a second bracket located within the handle housing and spaced apart along the length of the handle housing, the first bracket having a first slot and the second bracket having a second slot, one end of the circuit board inserted into the first slot and the other end into the second slot; and the second bracket being fixed to the handle housing, and the first and second light-emitting elements being fixed to the first bracket.

8. The handheld device of claim 1, further comprising two conductive members, a skin detection module, and a charging module, wherein one end of each conductive member is exposed from the handle housing or the phototherapy head, the other end connected to the skin detection module and the charging module respectively, forming a skin detection circuit and a charging circuit.

9. A handheld device comprising:
a handle housing comprising a rotating portion disposed at one end of the handle housing, and a mounting portion disposed at the other end of the handle housing;
a circuit board disposed within the handle housing;
at least one stimulation element disposed within the mounting portion and electrically connected to the circuit board; and
a massage element disposed outside the handle housing, the massage element having a first mounting cavity, the first mounting cavity is open for insertion of the rotating portion rotatably engages with the first mounting cavity.

10. The handheld device of claim 9, wherein one of the rotating portions and the massage element is provided with a rotating shaft, and the other is provided with an axial hole, the rotating shaft having a shaft sleeve located within the axial hole.

11. The handheld device of claim 10, the rotating portion is provided with a screw hole, the rotating shaft having a threaded portion at one end and a stopper at the other end, the threaded portion screwed to the screw hole, and the stopper located on a side of the shaft hole away from the screw hole, with the radial dimension of the stopper being greater than the diameter of the shaft hole.

12. The handheld device of claim 10, wherein a retaining groove is provided on a side of the massage element away from the handle, the axial hole extends through a bottom wall of the retaining groove, a retaining portion is located within the retaining groove, and the handheld device further comprises a sealing member disposed in the retaining groove to cover the retaining portion.

13. The handheld device of claim 12, wherein a raised portion is provided on the bottom wall of the first mounting cavity, the axial hole extends through the raised portion and communicates with the first mounting cavity; and a retaining rib is provided on the end surface of the rotating portion, the retaining rib surrounds an outer circumference of the raised portion.

14. The handheld device of claim 9, wherein the handle housing is elongated and tapered in a direction from the rotating portion toward the mounting portion; and the mounting portion is elongated, with longitudinal direction of the mounting portion intersecting with the longitudinal direction of the handle housing.

15. The handheld device of claim 9, wherein the at least one stimulation element includes a light therapy component electrically connected to the circuit board, the mounting portion has a conductive surface with a second mounting cavity, and a light-transmitting member is disposed at the opening of the second mounting cavity, the light therapy component being disposed within the light-transmitting member.

16. The handheld device of claim 9, wherein the at least one stimulation element comprises two micro-current electrodes for providing microcurrent therapy.

17. A method for providing a therapy to a user, comprising:
providing a handheld device comprising a handle housing, a phototherapy head having a first light-transmitting surface and a second light-transmitting surface disposed opposite each other, at least one massage element rotatably coupled to the handle;
gripping the handle housing;
emitting light from a first light-emitting element toward the first light-transmitting surface and from a second light-emitting element toward the second light-transmitting surface;
bringing at least one of the light-transmitting surfaces into contact with or proximity to a target skin area; and
moving the massage element over the target skin area.

18. The method of claim 17, wherein the massage element is rotatably coupled to a rotating portion disposed at one end of the handle, the method comprising rotating the rotating portion to cause the massage element to move over the target skin area.

19. The method of claim 17, further comprising: providing one or more microcurrent electrodes in the handheld device and forming a closed circuit through skin to deliver microcurrent to the target area.

20. The method of claim 17, further comprising adjusting orientation of the handle to selectively bring either the first or second light-transmitting surface into contact with the skin without repositioning the massage element.
